(19) United States
(12) Patent Application Publication
(10) Pub. No.: US 2026/0216418 A1
(43) Pub. Date: Jul. 30, 2026

(54) DEVICE AND SYSTEM FOR NEGATIVE PRESSURE WOUND THERAPY

(71) Applicant: Solventum Intellectual Properties Company, Maplewood, MN (US)

(72) Inventors: Dominic NOLAN, Bracknell (GB); Benjamin A. PRATT, Poole (GB); Robert HOWARD, Westmeath (GB)

(21) Appl. No.: 19/145,817

(22) PCT Filed: Jan. 9, 2024
(86) PCT No.: PCT/IB2024/050193

§ 371 Date: Jul. 3, 2025

Related U.S. Application Data
(60) Provisional application No. 63/438,056, filed on Jan. 10, 2023.

(51) Int. Cl. A61M 1/00

(52) U.S. Cl. CPC A61M 1/964; A61M 1/966

(57) ABSTRACT

A device for interfacing a negative pressure source with a dressing disposed on a wound site includes a housing. The housing includes a cavity including an opening configured to fluidly communicate with the dressing. The housing further includes a housing port extending through the housing and fluidly communicating with the cavity. The housing port is configured to fluidly couple the cavity with the negative pressure source. The device further includes a filter at least partially covering the cavity of the housing and facing the opening of the housing. The filter is air permeable. The device further includes a chamber defined between the housing and the filter opposite to the opening. An air flow between the chamber and the opening occurs through the filter. The device further includes a sensor received within the chamber and configured to sense at least one parameter associated with the wound site.

### CROSS-REFERENCE TO RELATED APPLICATIONS

This application is a U.S. National Stage Entry of PCT International Application No. PCT/IB2024/050193, filed Jan. 9, 2024, which claims the benefit of priority to U.S. Provisional Application No. 63/438,056, filed on Jan. 10, 2023, which is incorporated herein by reference in its entirety.

### TECHNICAL FIELD

The present disclosure relates generally to a device and a system for negative pressure wound therapy.

### BACKGROUND

Negative pressure wound therapy (NPWT) systems are embodied as sealed wound-care systems particularly indicated for chronic persistent wounds and/or complicated wounds. Specifically, NPWT systems involve application of a pressure that is reduced relative to the surroundings (commonly referred to as “negative pressure”) to the wound for promoting wound healing. Recent advancements in NPWT systems involve aspirating the wound together with provision of additional fluid for irrigating and/or cleansing the wound. Thereafter, a wound fluid including both wound exudates and the irrigation fluid is then drawn off by an aspiration means.

NPWT systems typically include a therapy unit that is in fluid communication with a wound site via a wound dressing applied on the wound site. Sensors (e.g., pressure sensors, temperature sensors, etc.) are typically mounted on the wound dressing and positioned in proximity to the wound site and/or the wound fluid for capturing parameters associated with the wound site and/or the wound fluid. However, such sensors are subjected to conditions that may inhibit their intended functionality. This may occur because of build-up of the wound exudates or the wound fluid on the sensor. This may lead to inaccurate readings that may negatively impact clinical outcomes.

### SUMMARY

Generally, the present disclosure relates to a device and a system for negative pressure wound therapy.

In a first aspect, the present disclosure provides a device for interfacing a negative pressure source with a dressing disposed on a wound site. The device includes a housing. The housing includes a cavity including an opening configured to fluidly communicate with the dressing. The housing further includes a housing port extending through the housing and fluidly communicating with the cavity. The housing port is configured to fluidly couple the cavity with the negative pressure source. The device further includes a filter at least partially covering the cavity of the housing and facing the opening of the housing. The filter is air permeable. The device further includes a chamber defined between the housing and the filter opposite to the opening. An air flow between the chamber and the opening occurs through the filter. The device further includes a sensor received within the chamber and configured to sense at least one parameter associated with the wound site.

In a second aspect, the present disclosure provides a system for negative pressure therapy of a wound site. The system includes a dressing disposed on the wound site and a negative pressure source. The system further includes a device for interfacing the negative pressure source with the dressing. The device includes a housing disposed on the dressing. The housing includes a cavity including an opening fluidly communicating with the dressing. The housing further includes a housing port extending through the housing and fluidly communicating with the cavity. The housing port fluidly couples the cavity with the negative pressure source. The device further includes a filter at least partially covering the cavity of the housing and facing the opening of the housing. The filter is air permeable. The device further includes a chamber defined between the housing and the filter opposite to the opening. An air flow between the chamber and the opening occurs through the filter. The device further includes a sensor received within the chamber and configured to sense at least one parameter associated with the wound site.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 illustrates a schematic view of a system for negative pressure therapy of a wound site, according to an embodiment of the present disclosure;

FIGS. 2A and 2B illustrate schematic top and bottom perspective views, respectively, of a device of the system of FIG. 1, according to an embodiment of the present disclosure;

FIGS. 3A and 3B illustrate schematic top and bottom partial exploded perspective views, respectively, of the device, according to an embodiment of the present disclosure;

FIG. 4 illustrates a schematic sectional view of the device taken along a section line A-A′ shown in FIG. 2A, according to an embodiment of the present disclosure;

FIG. 5A illustrates a schematic sectional perspective view of the device taken along a section line B-B′ shown in FIG. 2A, according to an embodiment of the present disclosure;

FIG. 5B illustrates a schematic exploded perspective view of the device of FIG. 5A, according to an embodiment of the present disclosure;

FIG. 6A illustrates a schematic sectional perspective view of the device taken along the section line B-B′ shown in FIG. 2A, according to another embodiment of the present disclosure;

FIG. 6B illustrates a schematic exploded perspective view of the device of FIG. 6A, according to an embodiment of the present disclosure;

FIG. 7A illustrates a schematic sectional view of the device taken along a section line C-C′ shown in FIG. 2A, according to an embodiment of the present disclosure;

FIG. 7B illustrates a schematic sectional view of the device taken along the section line C-C′ shown in FIG. 2A, where a pressure difference across a check valve is below a threshold pressure difference, according to an embodiment of the present disclosure;

FIG. 8A illustrates a schematic sectional view of the device taken along the section line C-C′ shown in FIG. 2A, according to another embodiment of the present disclosure;

FIG. 8B illustrates a schematic sectional view of the device taken along the section line C-C′ shown in FIG. 2A, where a pressure difference across a check valve is above a threshold pressure difference, according to an embodiment of the present disclosure;

FIGS. 9A and 9B illustrate schematic top and bottom perspective views, respectively, of the device, according to another embodiment of the present disclosure;

FIGS. 10A and 10B illustrate schematic top and bottom exploded perspective views, respectively, of the device of FIGS. 9A and 9B, according to an embodiment of the present disclosure;

FIG. 11 illustrates a schematic sectional view of the device taken along a section line D-D′ shown in FIG. 9A, according to an embodiment of the present disclosure;

FIG. 12 illustrates a schematic front exploded perspective view of the device of FIGS. 9A and 9B, according to an embodiment of the present disclosure;

FIG. 13 illustrates a schematic sectional view of the device taken along a section line E-E′ shown in FIG. 9A, according to an embodiment of the present disclosure;

FIG. 14A illustrates a schematic partial cross-sectional view of a filter, according to an embodiment of the present disclosure; and

FIG. 14B illustrates a schematic partial cross-sectional view of the filter, according to another embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

Unless specified or limited otherwise, the terms “attached,” “connected,” “coupled”, and variations thereof, are used broadly and encompass both direct physical connections, or indirect physical connections between two or more components that are connected together by one or more additional components. For example, a first component may be coupled to a second component by being directly connected together or by being connected by a third component. In some examples, coupling, connection, and attachment may also include mechanical, thermal, electrical, or chemical coupling (such as a chemical bond) in some contexts.

As used herein, the terms “layer,” “sheet,” and “dressing,” or variations thereof, are used to describe an article having a thickness that is small relative to its length and width.

As used herein, the term “negative pressure” generally refers to a pressure lower than a local ambient pressure in a local environment outside of a sealed treatment environment provided by a dressing at a wound site. In many cases, the local ambient pressure may also be the atmospheric pressure at which the wound site is located. Alternatively, the pressure may be less than a hydrostatic pressure associated with a tissue at the wound site. A reference to an increase in the negative pressure typically refers to a decrease in an absolute pressure, while a decrease in the negative pressure typically refers to an increase in the absolute pressure.

As used herein, the term “wound” may include, for example, chronic, acute, traumatic, subacute, closed surgical wounds, or dehiscence wounds, partially thick burns, ulcers (such as, diabetic, compressive, or venous insufficiency ulcers), flaps, and grafts. The term may also include an open abdomen area of a patient.

As used herein, the term “wound site” may include a tissue site, such as, bone tissue, adipose tissue, muscle tissue, nerve tissue, skin tissue, vascular tissue, connective tissue, cartilage, tendons, or ligaments. The term “wound site” may also refer to an area of a tissue that is not necessarily a wound or a defect but may be desired to add or promote additional tissue growth. For example, negative pressure therapy can be used at a particular tissue area to grow additional tissue that may be harvested or transplanted to another tissue site. The wound site may also include an area wherein a surgical incision has been previously performed.

Conventionally, a negative pressure wound therapy (NPWT) system includes a therapy unit that is in fluid communication with a wound site via a wound dressing applied to the wound site. The therapy unit is configured to provide negative pressure wound therapy by reducing a pressure at the wound site. The therapy unit may draw a vacuum (relative to atmospheric pressure) at the wound site by removing wound exudate, air, and other fluids from the wound. The therapy unit may also extract fluids from the wound site that may be previously delivered to the wound site, such as, for example, a cleansing fluid, a prescribed fluid, a medicated fluid, an antibiotic fluid, or any other type of fluid which may be delivered to the wound site for therapeutic purposes.

Sensors are typically mounted on the wound dressing and positioned in proximity to the wound site and/or the fluids. However, such sensors are subjected to conditions that may inhibit their intended functionality. This may occur because of build-up of the wound exudate or the fluids on the sensor. This may lead to inaccurate readings that may negatively impact clinical outcomes.

The present disclosure provides a device for interfacing a negative pressure source with a dressing disposed on a wound site. The device includes a housing. The housing includes a cavity including an opening configured to fluidly communicate with the dressing. The housing further includes a housing port extending through the housing and fluidly communicating with the cavity. The housing port is configured to fluidly couple the cavity with the negative pressure source. The device further includes a filter at least partially covering the cavity of the housing and facing the opening of the housing. The filter is air permeable. The device further includes a chamber defined between the housing and the filter opposite to the opening. An air flow between the chamber and the opening occurs through the filter. The device further includes a sensor received within the chamber and configured to sense at least one parameter associated with the wound site.

The device of the present disclosure includes the filter at least partially covering the cavity of the housing and facing the opening of the housing. Further, the filter may prevent wound fluids from entering the chamber disposed within the cavity of the housing and defined between the housing and the filter opposite to the opening. The sensor is received within the chamber, thereby eliminating any direct contact of the sensor with the wound fluids. Thus, the device of the present disclosure may protect the sensor and enhance a service life of the sensor. Further, the device may enable the sensor to perform its intended functions accurately throughout the application of a negative pressure therapy to the wound site. In some cases, the sensor may be a pressure sensor. The filter being air permeable may allow the sensor to perform the intended functions while eliminating any direct contact with the wound fluids.

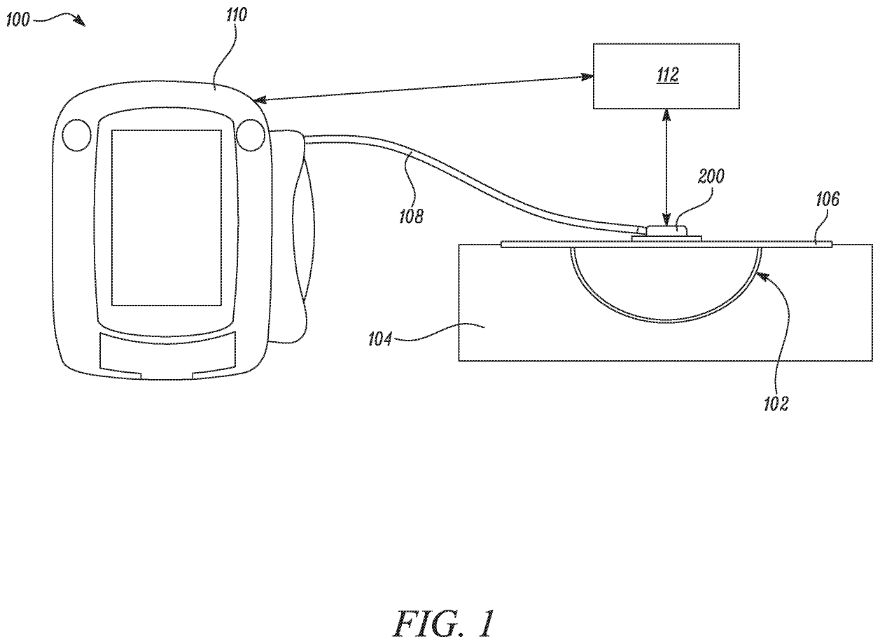

Referring now to Figures, FIG. 1 illustrates a schematic view of a system 100 for negative pressure therapy of a wound site 102. The system 100 may be used for healing the wound site 102 of a user (not shown). A skin 104 of the user includes the wound site 102 that is to be treated. In some examples, the system 100 may be used to provide negative pressure therapy, instillation of topical treatment solutions, and debridement to the wound site 102 for therapeutic purpose.

The system 100 includes a dressing 106 disposed on the wound site 102 and a negative pressure source 110. In some examples, the dressing 106 may cover the wound site 102, and may be adapted to seal the wound site 102 and create a therapeutic environment proximal to the wound site 102 for maintaining a negative pressure at the wound site 102. The dressing 106 is fluidly coupled to the negative pressure source 110 via a conduit 108 for applying the negative pressure at the wound site 102. In some examples, the dressing 106 is preferably detachable, and may be disposable, reusable, or recyclable. The term “conduit”, as used herein, broadly refers to a tube, a pipe, a hose, a conduit, or other structure with one or more lumina adapted to convey one or more fluids between two ends.

It should be understood that the dressing 106 is schematically shown for the purpose of illustration, and the dressing 106 may include components, such as a cover, a tissue interface, or both in some embodiments. In some examples, the tissue interface (not shown) of the dressing 106 may be generally adapted to contact the wound site 102. In some examples, the tissue interface may partially or completely fill the wound site 102 or may be placed over the wound site 102. In some examples, the cover (not shown) of the dressing 106 may provide a bacterial barrier and protection from physical trauma.

In some examples, the negative pressure source 110 may be a reservoir of air at a negative pressure or may be a manual or an electrically powered device (e.g., a negative pressure pump) that may reduce a pressure in a sealed volume, such as, a vacuum pump, a suction pump, or a wall suction port, available at many healthcare facilities, or a micro-pump, for example. The negative pressure source 110 may further include other components, such as, sensors, processing units, alarm indicators, memories, databases, software, display devices, and/or user interfaces that may further facilitate the negative pressure therapy.

In some examples, the negative pressure source 110 may also include a fluid instillation pump fluidly coupled to the wound site 102, separately or via the conduit 108. In some examples, the system 100 may further include features involving aspirating the wound site 102 together with provision of additional fluids for irrigating and/or cleansing the wound site 102. Thereafter, wound fluids including both wound exudates and the irrigation/cleansing fluids are drawn off by the negative pressure source 110, which may be collected in an exudate container disposed on the negative pressure source 110 or external to the negative pressure source 110. In some examples, the negative pressure applied at the wound site 102 may induce macrostrain and microstrain at the wound site 102, as well as remove the wound fluid from the wound site 102.

In some examples, the conduit 108 may allow the negative pressure generated by the negative pressure source 110 to be delivered to the wound site 102. The conduit 108 may be additionally used to collect the wound fluids from the wound site 102. In some examples, the conduit 108 may include one or more lumens adapted to convey a fluid between two ends. In some examples, the negative pressure source 110 system and the conduit 108 may be packaged as a single, integrated unit, such as a therapy system including all of the components shown in FIG. 1 that are fluidly coupled to the dressing 106.

In some examples, the system 100 further includes a controller 112 communicably coupled to the negative pressure source 110. In some examples, the controller 112 may control the negative pressure source 110 to generate the appropriate negative pressure through the negative pressure pump for the negative pressure therapy. In some examples, the controller 112 is external to the negative pressure source 110. Alternatively, the controller 112 may be disposed on the negative pressure source 110.

In some examples, the controller 112 may be embodied in a number of different ways. For example, the controller 112 may be embodied as various processing means, such as one or more of a microprocessor, or other processing elements, a coprocessor, or various other computing or processing devices, including integrated circuits, such as, for example, an ASIC (application specific integrated circuit), an FPGA (field programmable gate array), or the like. In some examples, the controller 112 may be configured to execute instructions stored in a memory. In some examples, the memory may be a cache memory, a system memory, or any other memory. Alternatively, or in addition, the memory may be integral with the controller 112, such as a cache or random-access memory.

As such, whether configured by hardware, or by a combination of hardware and software, the controller 112 may represent an entity (e.g., physically embodied in a circuitry—in the form of a processing circuitry) capable of performing operations according to some embodiments while configured accordingly. Thus, for example, when the controller 112 is embodied as an ASIC, FPGA, or the like, the controller 112 may have specifically configured hardware for conducting the operations described herein. Alternatively, as another example, when the controller 112 may be embodied as an executor of software instructions, the instructions may specifically configure the controller 112 to perform the operations described herein.

In some examples, the memory may be a main memory, a static memory, or a dynamic memory. The memory may include, but may not limited to, computer readable storage media, such as various types of volatile and non-volatile storage media, including, but not limited to, random access memory (RAM), read-only memory (ROM), programmable read-only memory, electrically programmable read-only memory (EPROM), electrically erasable programmable read-only memory (EEPROM), flash memory, magnetic tape or disk, optical media, solid-state memory array, and/or the like.

The system 100 further includes a device 200 for interfacing the negative pressure source 110 with the dressing 106. In some examples, the device 200 may facilitate coupling of the negative pressure source 110 to the dressing 106 and may enable the negative pressure generated by the negative pressure source 110 to be delivered to the wound site 102. In some examples, the controller 112 is further communicably coupled to the device 200.

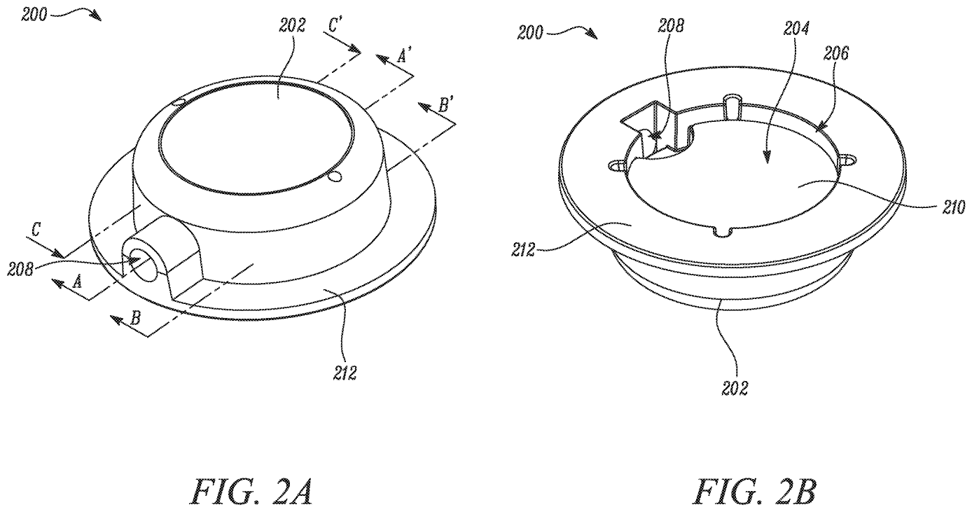

FIGS. 2A and 2B illustrate schematic top and bottom perspective views, respectively, of the device 200. Referring to FIGS. 1-2B, the device 200 includes a housing 202. The housing 202 includes a cavity 204 (shown in FIG. 2B) including an opening 206 (shown in FIG. 2B) configured to fluidly communicate with the dressing 106. In some examples, the opening 206 may interface with the dressing 106 in a fluid-tight manner enabling the opening 206 and the cavity 204 to fluidly communicate with the wound site 102. For example, the opening 206 may align with an opening of the dressing 106 that is in fluid communication with the wound site 102. In some examples, the housing 202 further includes a housing interface portion 212 engaging with the dressing 106 in a fluid-tight manner. In some examples, the housing 202 may be produced through molding.

The housing 202 further includes a housing port 208 extending through the housing 202 and fluidly communicating with the cavity 204. The housing port 208 (shown in FIG. 2A) is configured to fluidly couple the cavity 204 with the negative pressure source 110. Specifically, the housing port 208 is fluidly coupled to the conduit 108, thereby fluidly coupling the cavity 204 with the negative pressure source 110. Thus, the housing port 208 may deliver the negative pressure generated by the negative pressure source 110 to the wound site 102.

The device 200 further includes a filter 210 (shown in FIG. 2B) at least partially covering the cavity 204 of the housing 202 and facing the opening 206 of the housing 202. The filter 210 is air permeable. In some examples, the filter 210 covers at least a portion of the cavity 204 between the housing 202 and the opening 206. The filter 210 may be any means for separating substances, e.g., a means for separating a fluid from a gas.

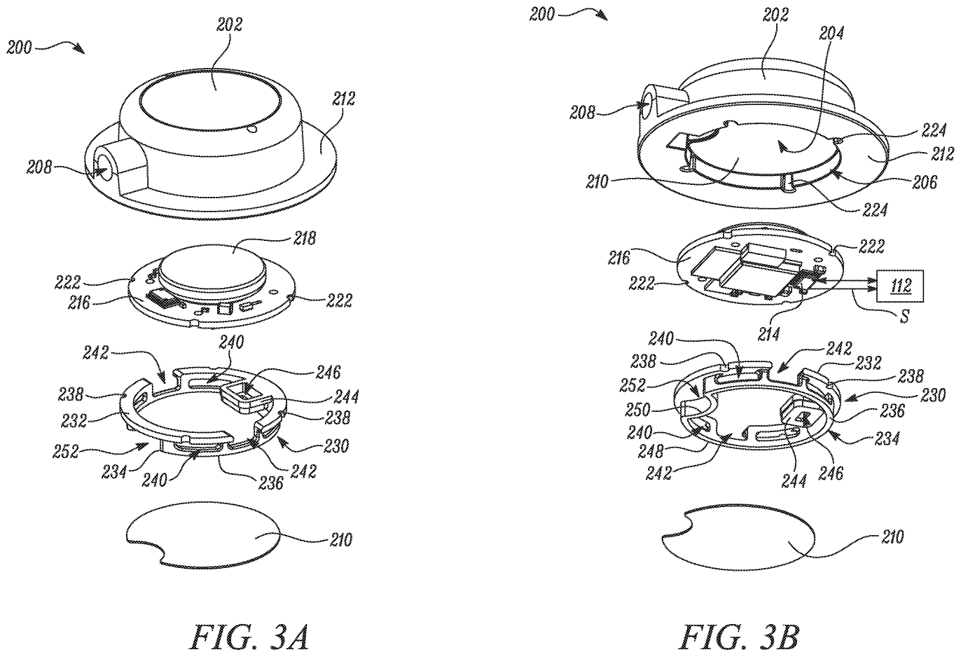

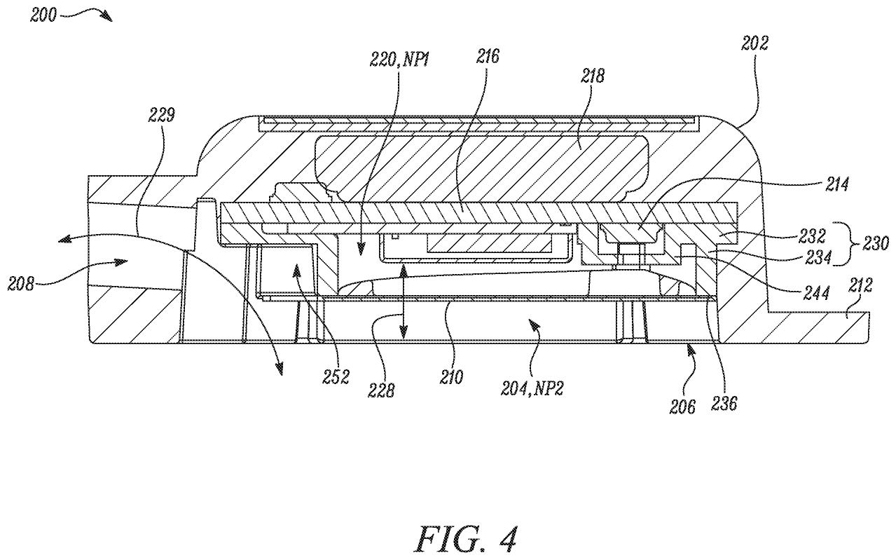

FIGS. 3A and 3B illustrate schematic top and bottom partial exploded perspective views, respectively, of the device 200. FIG. 4 illustrates a schematic sectional view of the device 200 taken along a section line A-A′ shown in FIG. 2A. Referring to FIGS. 3A-4, the device 200 further includes a chamber 220 (shown in FIG. 4) defined between the housing 202 and the filter 210 opposite to the opening 206. An air flow 228 (shown by a double-headed arrow in FIG. 4) between the chamber 220 and the opening 206 occurs through the filter 210.

The device 200 further includes a sensor 214 received within the chamber 220 and configured to sense at least one parameter associated with the wound site 102 (shown in FIG. 1). In general, the sensor 214 may be operable to detect or measure the at least one parameter, which may be a physical phenomenon or a property associated with the wound site 102 or the wound fluids.

In some examples, the sensor 214 is a pressure sensor. In some examples, the at least one parameter is a negative pressure NP1 within the chamber 220. In some examples, the controller 112 (shown in FIG. 1) is communicably coupled to the sensor 214. For example, the sensor 214 may detect the negative pressure NP1 within the chamber 220 and provide a signal S indicative of the negative pressure NP1 to the controller 112. As used herein, the term “pressure sensor” may generally refer to a transducer configured to measure a pressure in an enclosed space and convert the measurement to a signal indicative of the pressure measured. In some examples, the pressure sensor may be a piezoresistive strain gauge.

It should be understood that the sensor 214 may be any type of sensor, e.g., a pH sensor, a humidity sensor, a temperature sensor, a volatile organic compound (VOC) sensor, etc., that may detect the at least one parameter associated with the wound site 102 or the wound fluids.

In some examples, the device 200 further includes a printed circuit board 216 received within the chamber 220 and coupled to the housing 202. In some examples, the printed circuit board 216 is disposed between the filter 210 and the housing 202 opposite to the opening 206. In some examples, the sensor 214 is coupled to and disposed on the printed circuit board 216, such that the sensor 214 faces the filter 210. As used herein, the term “printed circuit board” is intended to cover various types of flexible and non-flexible substrates having a printed circuit pattern arranged on the surface thereof either with or without active or inactive circuit components also secured to the substrate.

In some examples, the device 200 further includes a power source 218 coupled to and disposed on the printed circuit board 216 opposite to the sensor 214. In some examples, the power source 218 is electrically coupled to the sensor 214. Thus, the sensor 214 may receive power from the power source 218 through the printed circuit board 216 for its operation. In some examples, the power source 218 may include one or more electrochemical cells, one or more capacitors, a rechargeable battery, a non-rechargeable battery, etc. In some examples, the printed circuit board 216 includes one or more circuit components mounted thereon.

In some examples, the device 200 further includes a carrier 230 received within the cavity 204 and removably coupled to the filter 210. In some examples, the carrier 230 at least partially engages with and is coupled to the housing 202. In some examples, the printed circuit board 216 is mounted on the carrier 230. In some examples, the carrier 230 may support the printed circuit board 216 and may allow the printed circuit board 216 to be mounted within the cavity 204 of the housing 202. In some examples, the sensor 214 may be first mounted on the printed circuit board 216 before mounting the printed circuit board 216 on the carrier 230. In some examples, the carrier 230 may be flexible or rigid. In alternative embodiments, the device 200 may not include the carrier 230 and the printed circuit board 216 may be directly coupled to the housing 202.

In some examples, the carrier 230 includes a base 232 engaging the printed circuit board 216 and a lateral wall 234 extending from the base 232 towards the opening 206. In some examples, the lateral wall 234 is continuous and surrounds the filter 210. In some examples, the lateral wall 234 sealingly engages the filter 210. Specifically, an end surface 236 (shown in FIG. 3B) of the lateral wall 234 sealingly engages the filter 210. In some examples, the lateral wall 234 includes a plurality of slots 240 (shown in FIGS. 3A and 3B) extending therethrough.

In some examples, the carrier 230, the housing 202, and the filter 210 together define the chamber 220 therebetween. In some examples, the filter 210 and the carrier 230 may prevent the wound fluids from entering the chamber 220 within the cavity 204 of the housing 202. Thus, the chamber 220 may eliminate any direct contact of the sensor 214 with the wound fluids. Therefore, the device 200 of the present disclosure may allow the sensor 214 to perform its intended functions accurately throughout the application of a negative pressure therapy to the wound site 102 (shown in FIG. 1). The filter 210 being air permeable may allow the sensor 214 (e.g., the pressure sensor) to perform the intended functions while eliminating any direct contact with the wound fluids. In some examples, the filter 210 may be a flexible or a rigid mesh filter, a fabric filter (woven or non-woven), a membrane filter, etc.

As shown in FIGS. 3A and 3B, in some examples, the base 232 further includes a plurality of base notches 238. In some examples, the printed circuit board 216 includes a plurality of board notches 222 corresponding to and at least partially aligned with the plurality of base notches 238. In some examples, the housing 202 further includes a plurality of housing recesses 224 corresponding to and communicating with the plurality of board notches 222 and the plurality of base notches 238. In some examples, the plurality of base notches 238, the plurality of board notches 222, and the plurality of housing recesses 224 may together receive a fastener (e.g., a screw) for fastening the carrier 230 and the printed circuit board 216 to the housing 202.

In some examples, the carrier 230 further includes a plurality of cutouts 242 extending through the base 232 and bounded by the lateral wall 234. In some examples, each of the plurality of cutouts 242 is disposed between adjacent slots 240 from the plurality of slots 240. In some examples, the plurality of cutouts 242 and the plurality of slots 240 may reduce an overall weight of the carrier 230.

In some examples, the carrier 230 further includes a sensor portion 244 extending inwards from the lateral wall 234 opposite to the housing 202 and including a sensor opening 246 at least partially aligned with the sensor 214. In some examples, the sensor portion 244 is disposed on the printed circuit board 216. In some examples, the sensor 214 may be at least partially received within the sensor portion 244 and configured to sense the at least one parameter through the sensor opening 246.

In some examples, the lateral wall 234 further includes a circular arc portion 248 (shown in FIG. 3B) and a notch portion 250 (shown in FIG. 3B) connected to the circular arc portion 248 and curving radially inwards relative to the circular arc portion 248. In some examples, the notch portion 250 defines a recess 252 facing the housing port 208. In some examples, the notch portion 250 may not include any slots.

In some examples, the notch portion 250 and the recess 252 may allow the housing port 208 to fluidly communicate with the opening 206 and the dressing 106 (shown in FIG. 1) while ensuring that the chamber 220 remains sealed at all times during application of the negative pressure to the wound site 102. In some examples, fluids may flow along a flow path 229 (shown by a curved double-headed arrow in FIG. 4) between the housing port 208 and the opening 206 away from the chamber 220. Thus, the negative pressure source 110 (shown in FIG. 1) may be fluidly coupled to the wound site 102 (shown in FIG. 1) through the conduit 108 (shown in FIG. 1), the housing port 208, the recess 252, and the opening 206.

In some examples, the negative pressure NP1 within the chamber 220 may be same as a negative pressure NP2 within the cavity 204 and the wound site 102 if the filter 210 is clean. Thus, the sensor 214 may be able to determine the negative pressure NP2 at the wound site 102 by detecting the negative pressure NP1 within the chamber 220. However, the negative pressure NP1 within the chamber 220 may differ from the negative pressure at the wound site 102 if the filter 210 is at least partially blocked.

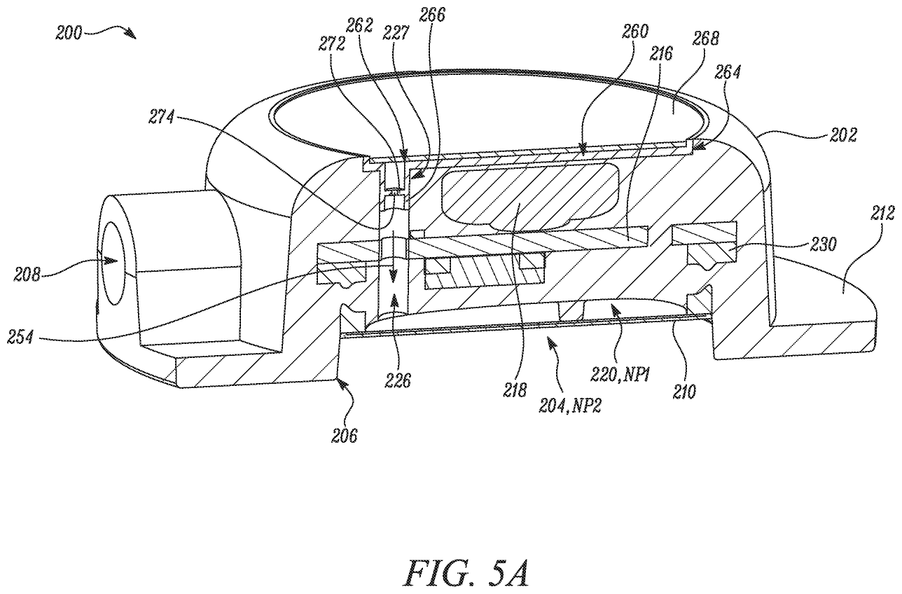

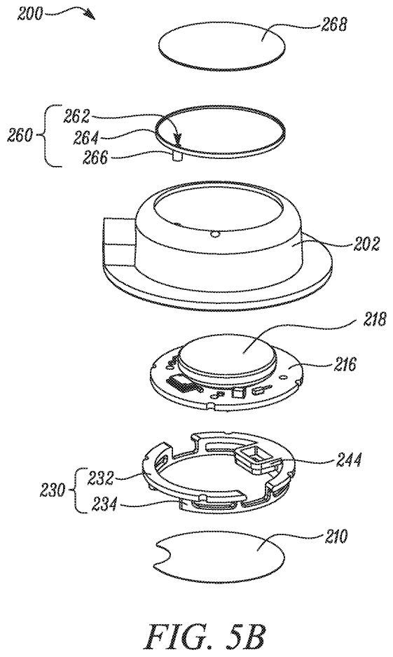

FIG. 5A illustrates a schematic sectional perspective view of the device 200 taken along a section line B-B′ shown in FIG. 2A. In some examples, the housing 202 further includes at least one leak path 226 extending therethrough and fluidly communicating with the chamber 220. In the illustrated embodiment of FIG. 5A, the at least one leak path 226 includes a single leak path 226. In some examples, the at least one leak path 226 may be molded into the housing 202.

In some examples, the at least one leak path 226 may fluidly communicate the chamber 220 with an ambient. For example, an end 227 of the at least one leak path 226 opposite to the filter 210 may be disposed in fluid communication with the ambient. Thus, the at least one leak path 226 may allow an air flow 254 to be received within the chamber 220 by virtue of the negative pressure NP1 within the chamber 220. The ambient air may then be received within the cavity 204 outside the chamber 220 after flowing through the filter 210 since the filter 210 is air permeable. Air flowing through the filter 210 may minimize blockages in the filter 210 that may otherwise occur due to build-up of the wound fluids and its constituents, such as proteins, on a surface of the filter 210 that is facing the opening 206, thereby reducing any build-up of contaminants and improving a service life of the filter 210. Further, this may effectively improve an accuracy and a performance of the sensor 214.

FIG. 5B illustrates a schematic exploded perspective view of the device 200 shown in FIG. 5A. Referring to FIGS. 5A and 5B, in some examples, the device 200 further includes an ambient carrier 260 coupled to the housing 202 and disposed opposite to the filter 210, such that the chamber 220 is disposed between the ambient carrier 260 and the filter 210. In some examples, the ambient carrier 260 includes at least one ambient opening 262 extending therethrough. In some examples, each of the at least one ambient opening 262 is disposed in fluid communication with the corresponding at least one leak path 226 (shown in FIG. 5A). In the illustrated embodiment of FIGS. 5A and 5B, the ambient carrier 260 includes a single ambient opening 262 disposed in fluid communication with the single leak path 226.

In some examples, the ambient carrier 260 further includes a main body 264. In some examples, the at least one ambient opening 262 extends through the main body 264. In some examples, the ambient carrier 260 further includes at least one hollow tube 266 extending from the main body 264. In some examples, each of the at least one hollow tube 266 is at least partially received within and disposed in fluid communication with the corresponding at least one leak path 226. In some examples, each of the at least one hollow tube 266 is disposed in fluid communication with the corresponding at least one ambient opening 262. In the illustrated embodiment of FIGS. 5A and 5B, the ambient carrier 260 includes a single hollow tube 266 disposed in fluid communication with the single leak path 226 and the single ambient opening 262.

In some examples, the ambient carrier 260 further includes a tube wall 272 (shown in FIG. 5A) disposed within each of the at least one hollow tube 266 and including an orifice 274 (shown in FIG. 5A) extending therethrough. In some examples, each of the at least one hollow tube 266 fluidly communicates the corresponding at least one leak path 226 with the corresponding at least one ambient opening 262 via the corresponding orifice 274. In some examples, the air flow 254 may pass through the orifice 274 of the tube wall 272 before entering the chamber 220. In some examples, the orifice 274 of the tube wall 272 of each of the at least one hollow tube 266 is small in dimensions and may allow a limited fixed leakage flow of air therethrough, i.e., the air flow 254. The fixed leakage flow of air may reduce the build-up of wound fluids on the surface of the filter 210 facing the opening 206. Further, the air flow 254 may not provide significant burden on the negative pressure source 110 (shown in FIG. 1).

In some examples, the device 200 further includes an ambient filter 268 disposed on the ambient carrier 260 opposite to the at least one leak path 226 and removably coupled to the housing 202. In some examples, the ambient filter 268 covers the at least one ambient opening 262 of the ambient carrier 260 and is exposed to the ambient. In some examples, the ambient filter 268 is air permeable. In some examples, the ambient filter 268 may be any porous structure with openings sized to make a desired separation, e.g., a mesh filter, a fabric filter (woven or non-woven), a membrane filter, etc. For example, the ambient filter 268 may eliminate any contaminants (such as dirt, suspended particles, etc.) in the ambient air before the air enters the at least one ambient opening 262. In some examples, the ambient filter 268 may be made of any suitable material, e.g., a polymer, a metal, an alloy, a ceramic.

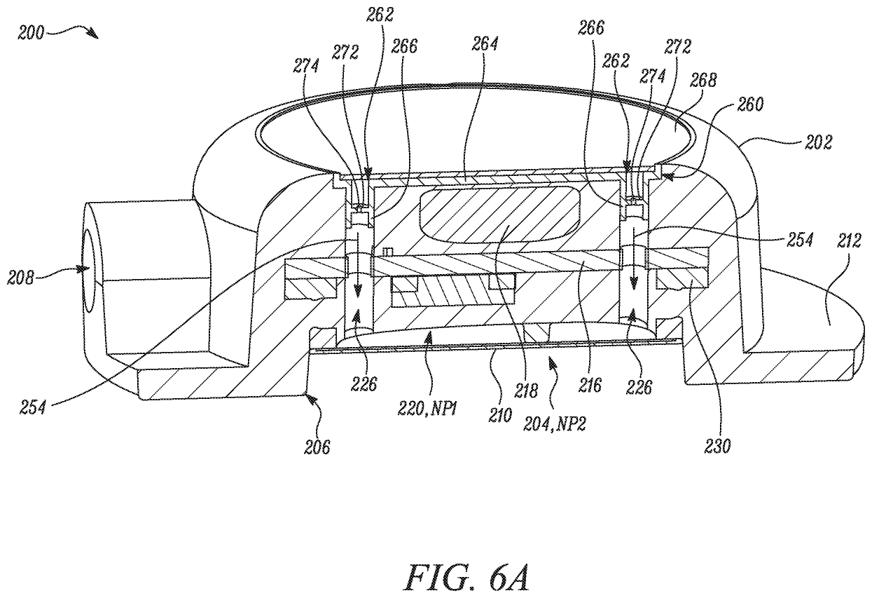

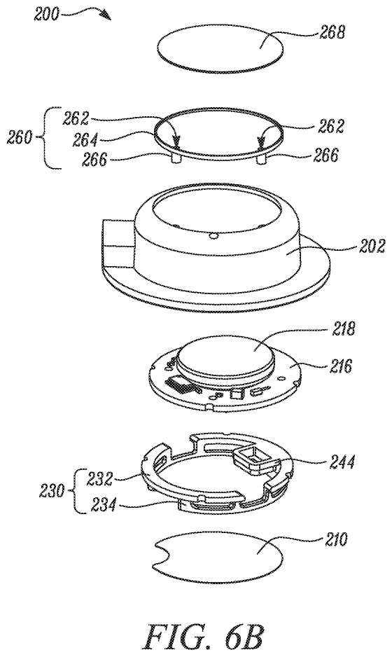

FIG. 6A illustrates a schematic sectional perspective view of the device 200 taken along the section line B-B′ shown in FIG. 2A, according to another embodiment of the present disclosure. FIG. 6B illustrates a schematic exploded perspective view of the device 200 of FIG. 6A. In the illustrated embodiment of FIGS. 6A and 6B, the at least one leak path 226 includes a plurality of leak paths 226 spaced apart from each other. Specifically, in FIGS. 6A and 6B, the plurality of leak paths 226 includes a pair of leak paths 226.

Further, the at least one ambient opening 262 includes a plurality of ambient openings 262 corresponding to the plurality of the plurality of leak paths 226. Specifically, each of the plurality of ambient openings 262 extends through the main body 264. Each of the plurality of ambient openings 262 is disposed in fluid communication with the corresponding leak path 226 from the plurality of leak paths 226. In some examples, the at least one hollow tube 266 includes a plurality of hollow tubes 266. Each of the plurality of hollow tubes 266 is at least partially received within and disposed in fluid communication with the corresponding leak path 226. In the illustrated embodiment of FIGS. 6A and 6B, the plurality of ambient openings 262 include a pair of ambient openings 262 corresponding to the pair of leak paths 226, and the plurality of hollow tubes 266 include a pair of hollow tubes 266 corresponding to the pair of leak paths 226.

In some examples, each of the plurality of hollow tubes 266 is disposed in fluid communication with the corresponding ambient opening 262. In some examples, the ambient carrier 260 further includes the tube wall 272 disposed within each of the plurality of hollow tubes 266 and including the orifice 274 extending therethrough. In some examples, the orifice 274 of the tube wall 272 of each of the plurality of hollow tubes 266 may allow the air flow 254 to pass therethrough. Thus, the plurality of leak paths 226 may enable the air flow 254 from the orifices 274 of the tube walls 272 to be passed through a wider area of the filter 210, thereby effectively eliminating blockages in the filter 210.

In some examples, the device 200 further includes the ambient filter 268 disposed on the ambient carrier 260 opposite to the plurality of leak paths 226 and removably coupled to the housing 202. In some examples, the ambient filter 268 covers the plurality of ambient openings 262 of the ambient carrier 260 and is exposed to the ambient.

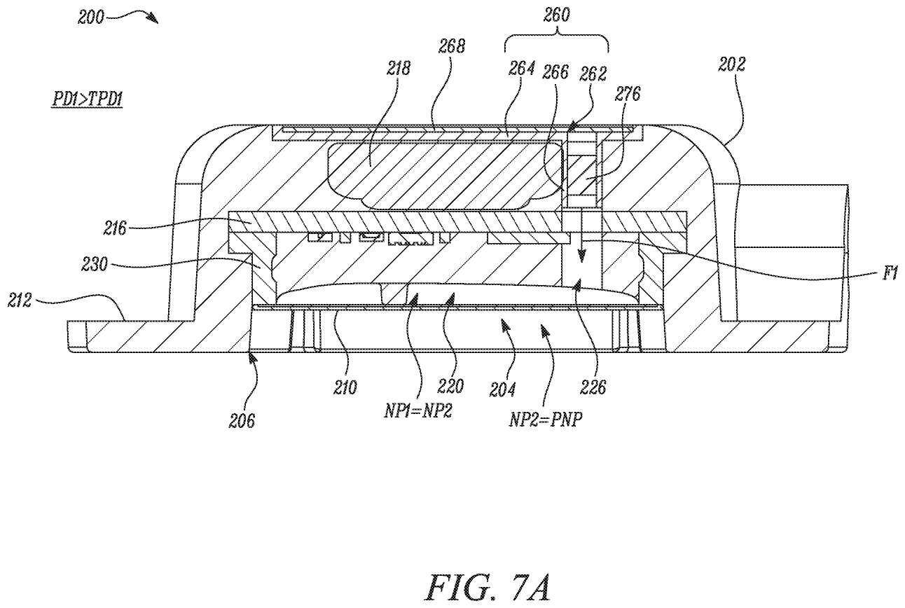

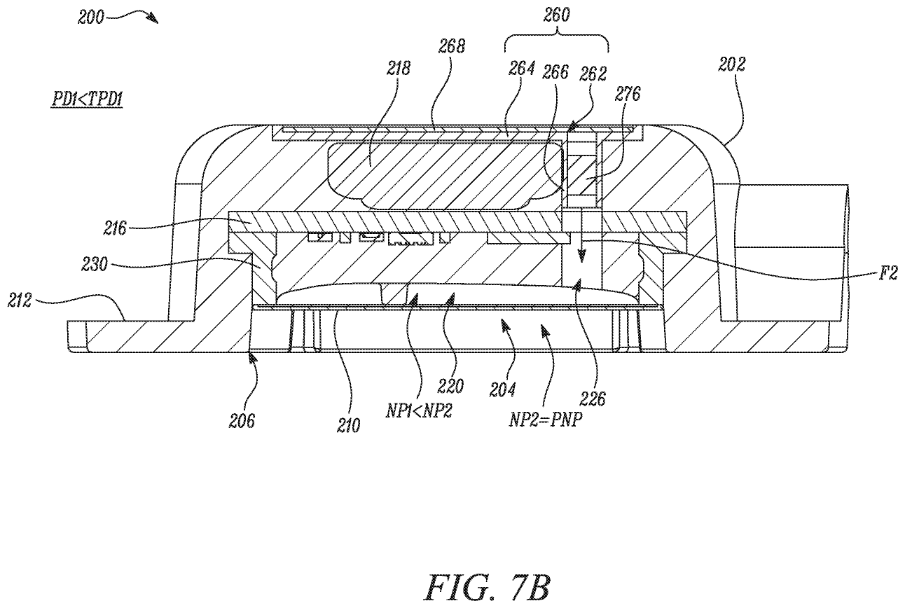

FIG. 7A illustrates a schematic sectional view of the device 200 taken along a section line C-C′ shown in FIG. 2A, according to another embodiment of the present disclosure. In some examples, the housing 202 further includes the at least one leak path 226 extending therethrough and fluidly communicating with the chamber 220. In the illustrated embodiment of FIG. 7A, the at least one leak path 226 includes a single leak path 226. In some examples, the at least one leak path 226 may fluidly communicate the chamber 220 with the ambient.

In some examples, the device 200 further includes the ambient carrier 260 coupled to the housing 202 and including the at least one ambient opening 262 extending therethrough. In some examples, each of the at least one ambient opening 262 is disposed in fluid communication with the corresponding at least one leak path 226. In some examples, the ambient carrier 260 further includes the main body 264 and the at least one hollow tube 266 extending from the main body 264. In some examples, each of the at least one hollow tube 266 is at least partially received within and disposed in fluid communication with the corresponding at least one leak path 226. In some examples, each of the at least one hollow tube 266 is disposed in fluid communication with the corresponding at least one ambient opening 262.

In the illustrated embodiment of FIG. 7A, the device 200 further includes a check valve 276 disposed in each of the at least one hollow tube 266. The check valve 276 is schematically shown in FIG. 7A for the purpose of illustration. In some examples, the check valve 276 may be any type of check valve that allows a unidirectional flow of fluid, e.g., a duckbill valve. In some examples, the check valve 276 allows a leakage flow F1 therethrough when a pressure difference PD1 across the check valve 276 is greater than or equal to a threshold pressure difference TPD1. Therefore, the check valve 276 may not fully restrict fluid flow therethrough in a closed state and may allow the leakage flow F1 in the closed state. In the example described below, the threshold pressure difference TPD1 is equal to 75 mmHg.

In some examples, the controller 112 (shown in FIG. 1) may maintain the negative pressure NP2 within the cavity 204 and the dressing 106 (shown in FIG. 1) equal to a predetermined negative pressure PNP for therapeutic purposes using the negative pressure source 110 (shown in FIG. 1). In some examples, the predetermined negative pressure PNP is equal to 125 mmHg. The negative pressure NP1 within the chamber 220 is equal to the negative pressure NP2 within the cavity 204, i.e., 125 mmHg, since the filter 210 is air permeable. Thus, the pressure difference PD1 across the check valve 276, i.e., a difference between the negative pressure NP1 within the chamber 220 and an ambient pressure (e.g., 0 mmHg) is 125 mmHg. In such cases, the check valve 276 allows the leakage flow F1 therethrough since the pressure difference PD1 across the check valve 276 is greater than or equal to the threshold pressure difference TPD1 (i.e., 75 mmHg).

In some examples, the check valve 276 may be a normally opened check valve. In other words, the check valve 276 may allow unrestricted flow of air therethrough when the pressure difference PD1 across the check valve 276 is zero or of very low magnitude. In some examples, at the beginning of the negative pressure therapy, the pressure difference PD1 across the check valve 276 may be zero since the negative pressure NP1 inside the chamber 220 is substantially equal to the ambient pressure (i.e., 0 mmHg). As the negative pressure NP1 inside the chamber 220 increases due to an increase in the negative pressure NP2 inside the cavity 204 and the dressing 106 (delivered by the negative pressure source 110), the pressure difference PD1 across the check valve 276 increases.

Subsequently, the check valve 276 substantially closes, but allows the leakage flow F1 therethrough as the pressure difference PD1 across the check valve 276 becomes greater than or equal to the threshold pressure difference TPD1 (i.e., 75 mmHg). In some examples, the leakage flow F1 may be comparable with the air flow 254 through the orifice 274 (shown in FIGS. 5A and 6A). Thus, the leakage flow F1 may not provide significant burden on the negative pressure source 110 (shown in FIG. 1). In some examples, the leakage flow F1 may be received within the chamber 220 from the ambient by virtue of the negative pressure NP1 within the chamber 220 and the operation of the check valve 276.

In the illustrated embodiment of FIG. 7A, the pressure difference PD1 across the check valve 276 is greater than or equal to the threshold pressure difference TPD1 (i.e., 75 mmHg). However, the filter 210 may get blocked due to the build-up of the wound exudates and the wound fluids on the surface of the filter 210 that is facing the opening 206. This may reduce the negative pressure NP1 inside the chamber 220 due to the leakage flow F1 across the check valve 276. Subsequently, the negative pressure NP1 within the chamber 220 may fall such that the pressure difference PD1 across the check valve 276 is lower than the threshold pressure difference TPD1 (i.e., 75 mmHg). This is shown in FIG. 7B.

FIG. 7B illustrates a schematic sectional view of the device 200 taken along the section line C-C′ shown in FIG. 2A, where the pressure difference PD1 across the check valve 276 is below the threshold pressure difference TPD1 (i.e., 75 mmHg). In some examples, the check valve 276 opens and allows a unidirectional flow F2 from the corresponding at least one ambient opening 262 to the chamber 220 when the pressure difference PD1 across the check valve 276 is below the threshold pressure difference TPD1 (i.e., 75 mmHg). As the negative pressure NP1 reduces within the chamber 220, the check valve 276 opens to allow the unidirectional flow F2 from the corresponding at least one ambient opening 262 to the chamber 220. This occurs when the pressure difference PD1 across the check valve 276 reduces below the threshold pressure difference TPD1 (i.e., 75 mmHg).

In some examples, the unidirectional flow F2 may be larger than the leakage flow F1 (shown in FIG. 7A), thereby allowing a large flow of air to be received within the chamber 220. This may remove large blockages in the filter 210 due to the large flow of the air. In some examples, the unidirectional flow F2 may be larger than the leakage flow F1 (shown in FIG. 7A) by a factor of at least 10. After the blockages are eliminated, the negative pressure NP1 within the chamber 220 may be restored back to the predetermined negative pressure PNP (i.e., 125 mmHg), thereby substantially closing the check valve 276 and allowing the leakage flow F1 therethrough as the pressure difference PD1 across the check valve 276 is greater than or equal to the threshold pressure difference TPD1 (i.e., 75 mmHg). This cycle may repeat as the filter 210 gets blocked again. Thus, the check valve 276 may reduce a risk of blockage in the filter 210 (through the leakage flow F1 shown in FIG. 7A) and provide the ability to allow a higher flow when required.

It should be understood that the values for the threshold pressure difference TPD1 (i.e., 75 mmHg) and the predetermined negative pressure PNP (i.e., 125 mmHg) described above are exemplary in nature and the values may vary based on application requirements.

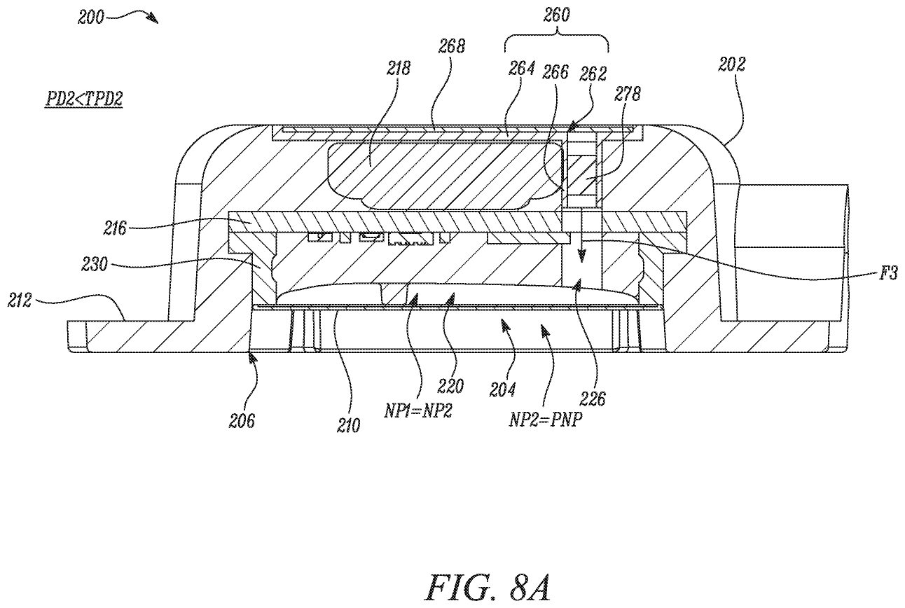

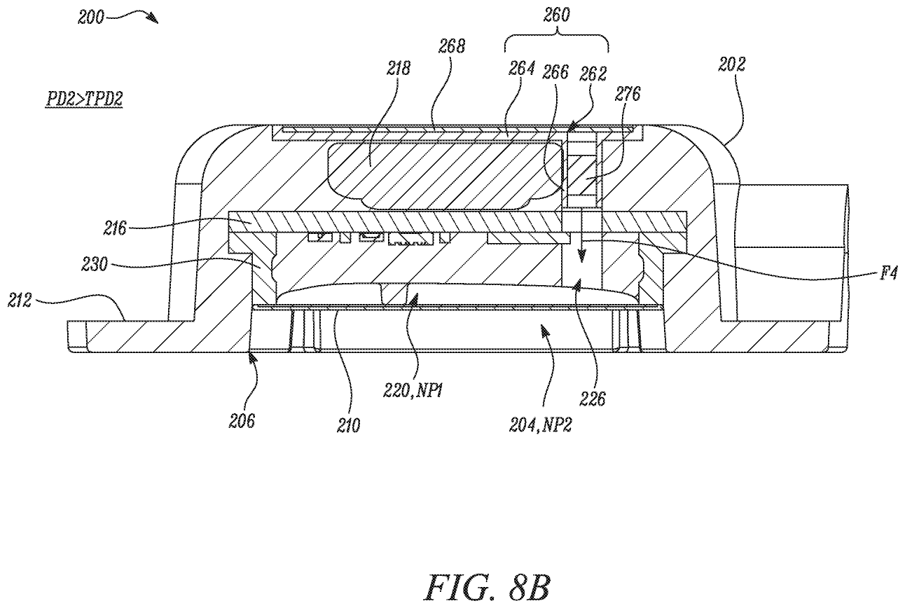

FIG. 8A illustrates a schematic sectional view of the device 200 taken along the section line C-C′ shown in FIG. 2A, according to another embodiment of the present disclosure. In the illustrated embodiment of FIG. 8A, the device 200 includes a check valve 278 disposed in each of the at least one hollow tube 266, instead of the check valve 276 shown in FIGS. 7A and 7B. The check valve 278 is schematically shown in FIG. 8A for the purpose of illustration. In some examples, the check valve 278 allows a leakage flow F3 therethrough when a pressure difference PD2 across the check valve 278 is less than or equal to a threshold pressure difference TPD2. Therefore, the check valve 278 may not fully restrict fluid flow therethrough in a closed state and may allow the leakage flow F3 in the closed state. In the example described below, the threshold pressure difference TPD2 is equal to 150 mmHg.

In some examples, the controller 112 (shown in FIG. 1) may maintain the negative pressure NP2 within the cavity 204 and the dressing 106 (shown in FIG. 1) equal to the predetermined negative pressure PNP for therapeutic purposes using the negative pressure source 110 (shown in FIG. 1). The negative pressure NP1 within the chamber 220 is equal to the negative pressure NP2 within the cavity 204, i.e., 125 mmHg, since the filter 210 is air permeable. Thus, the pressure difference PD2 across the check valve 278 is 125 mmHg. In such cases, the check valve 278 allows the leakage flow F3 therethrough since the pressure difference PD2 across the check valve 278 is less than or equal to the threshold pressure difference TPD2 (i.e., 150 mmHg).

In some examples, the check valve 278 may be a normally closed check valve. In other words, the check valve 278 may be closed when the pressure difference PD2 across the check valve 278 is zero or of very low magnitude. However, the check valve 278 may allow a very low leakage flow therethrough (e.g., the leakage flow F3) even in the closed state. In some examples, at the beginning of the negative pressure therapy, the pressure difference PD2 across the check valve 278 may be zero since the negative pressure NP1 inside the chamber 220 is substantially equal to the ambient pressure (i.e., 0 mmHg).

As the negative pressure NP1 inside the chamber 220 increases due to the increase in the negative pressure NP2 inside the cavity 204 (delivered by the negative pressure source 110), the pressure difference PD2 across the check valve 278 increases. The check valve 278 may allow the leakage flow F3 therethrough even when the negative pressure NP2 inside the cavity 204 or the negative pressure NP1 inside the chamber 220 are equal to the predetermined negative pressure PNP (i.e., 125 mmHg). Here, the pressure difference PD2 across the check valve 278 is less than or equal to the threshold pressure difference TPD2 (i.e., 150 mmHg).

In some examples, the leakage flow F3 may be comparable with the air flow 254 through the orifice 274 (shown in FIGS. 5A and 6A). Thus, the leakage flow F3 may not provide significant burden on the negative pressure source 110 (shown in FIG. 1). In some examples, the leakage flow F3 may be received within the chamber 220 from the ambient by virtue of the negative pressure NP1 within the chamber 220 and the operation of the check valve 278.

However, the filter 210 may get blocked due to the build-up of the wound exudates and the wound fluids on the surface of the filter 210 that is facing the opening 206. This may reduce the negative pressure NP1 within the chamber 220 due to the leakage flow F3 across the check valve 278. In some examples, the controller 112 is further configured to determine the negative pressure NP1 within the chamber 220 based on the signal S (shown in FIG. 3B) received from the sensor 214 (shown in FIGS. 3B and 4). In some examples, the controller 112 is further configured to determine that the filter 210 is blocked when the negative pressure NP1 within the chamber 220 falls below the predetermined negative pressure PNP (i.e., 125 mmHg).

In some examples example, the controller 112 may determine that the filter 210 is blocked by determining a difference between a negative pressure generated by the negative pressure source 110 (or a pump pressure) and the negative pressure NP1 within the chamber 220 sensed by the sensor 214 based on the signal S received from the sensor 214. If the filter 210 gets blocked, the negative pressure NP1 within the chamber 220 may be lower than the negative pressure generated by the negative pressure source 110.

FIG. 8B illustrates a schematic sectional view of the device 200 taken along the section line C-C′ shown in FIG. 2A, where the pressure difference PD2 across the check valve 278 is above the threshold pressure difference TPD2 (i.e., 150 mmHg). In some examples, upon detection of the blockage in the filter 210, the controller 112 is further configured to control the negative pressure source 110 to increase the negative pressure NP2 within the dressing 106 above the predetermined negative pressure PNP (i.e., 125 mmHg), such that the pressure difference PD2 across the check valve 278 is greater than the threshold pressure difference TPD2 (i.e., 150 mmHg). In other words, the controller 112 is further configured to control the negative pressure source 110 such that negative pressure NP2 within the dressing 106 is increased, thereby increasing the negative pressure NP1 within the chamber 220. This may increase the pressure difference PD2 across the check valve 278.

In some examples, the check valve 278 opens and allows a unidirectional flow F4 from the corresponding at least one ambient opening 262 to the chamber 220 when the pressure difference PD2 across the check valve 278 is above the threshold pressure difference TPD2 (i.e., 150 mmHg). For example, as the negative pressure NP1 within the chamber 220 increases such that the pressure difference PD2 across the check valve 278 is above the threshold pressure difference TPD2 (i.e., 150 mmHg), the check valve 278 opens and allows the unidirectional flow F4 from the corresponding at least one ambient opening 262 to the chamber 220.

In some examples, the unidirectional flow F4 may be larger than the leakage flow F3, thereby allowing a large flow of air to be received within the chamber 220. This may remove large blockages in the filter 210. In some examples, the unidirectional flow F4 may be larger than the leakage flow F3 (shown in FIG. 8A) by a factor of at least 10. The controller 112 may then determine that the filter 210 is free from any blockages based on the pump pressure and the signal S received from the sensor 214, the signal S being indicative of the negative pressure NP1 within the chamber 220.

After the blockages are eliminated, the controller 112 may again control the negative pressure source 110 to deliver the predetermined negative pressure PNP to the device 200, thereby reducing the pressure difference PD2 across the check valve 278. Subsequently, the check valve 278 allows the leakage flow F3 (shown in FIG. 8A) therethrough when the pressure difference PD2 across the check valve 278 is less than or equal to the threshold pressure difference TPD2 (i.e., 150 mmHg). Thus, the check valve 278 may reduce a risk of blockage in the filter 210 (through the leakage flow F3 shown in FIG. 8A) and provide the ability to allow a higher flow when required.

In some examples, the device 200 may further include alternative features that may allow the sensor 214 (shown in FIGS. 3B and 4) to perform its intended functions accurately for a prolonged period of time or may enhance the service life of the filter 210. For example, the device 200 may include a plurality of switching chambers. Each switching chamber may include a new filter which has not been exposed to the wound fluids. When one of the plurality of switching chambers does not allow the sensor 214 to sense the at least one parameter accurately due to a blockage in the corresponding filter, an alternative chamber with a new filter may be opened allowing unblocked flow of fluids.

Alternatively, the filter 210 may include a plurality of rolls of filters. One or more rolls of filters from the plurality of rolls of filters may be unrolled once blocked. In some examples, the device 200 may include one or more wipers intended to clean a surface of the filter 210 that is facing the opening 206. In some examples, the device 200 may include arrangements to clean the filter 210 through a cleaning fluid that is instilled into the wound site 102 (shown in FIG. 1).

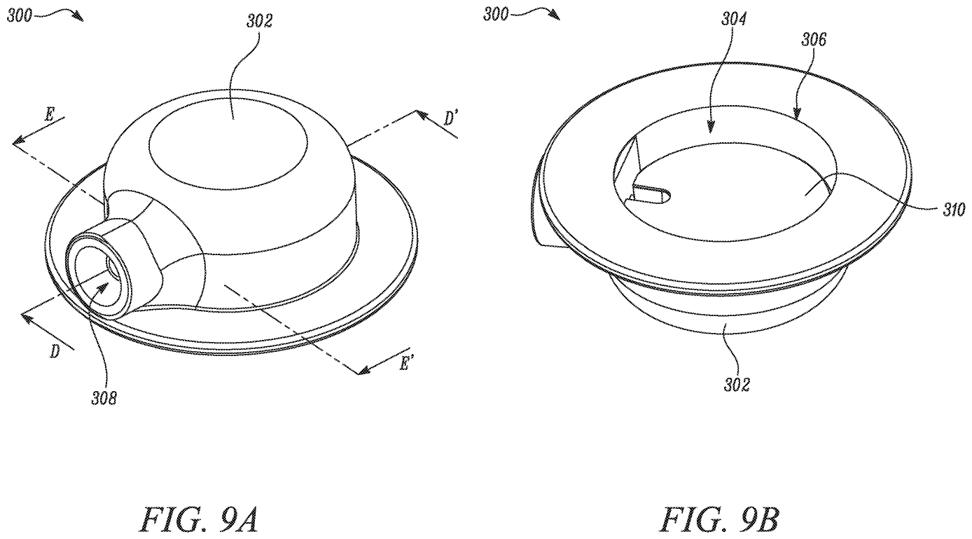

FIGS. 9A and 9B illustrate schematic top and bottom perspective views, respectively, of a device 300. The device 300 may be functionally similar to the device 200 shown in FIGS. 2A-8B, with same components being referred to by equivalent reference numerals. Referring to FIGS. 9A and 9B, the device 300 includes the housing 302. The housing 302 includes a cavity 304 (shown in FIG. 9B) including an opening 306 (shown in FIG. 9B) configured to fluidly communicate with a dressing (e.g., the dressing 106 shown in FIG. 1).

The housing 302 further includes a housing port 308 (shown in FIG. 9A) extending through the housing 302 and fluidly communicating with the cavity 304. The housing port 308 is configured to fluidly couple the cavity 304 with a negative pressure source (e.g., the negative pressure source 110 shown in FIG. 1). The device 300 further includes a filter 310 at least partially covering the cavity 304 of the housing 302 and facing the opening 306 of the housing 302. The filter 310 is air permeable.

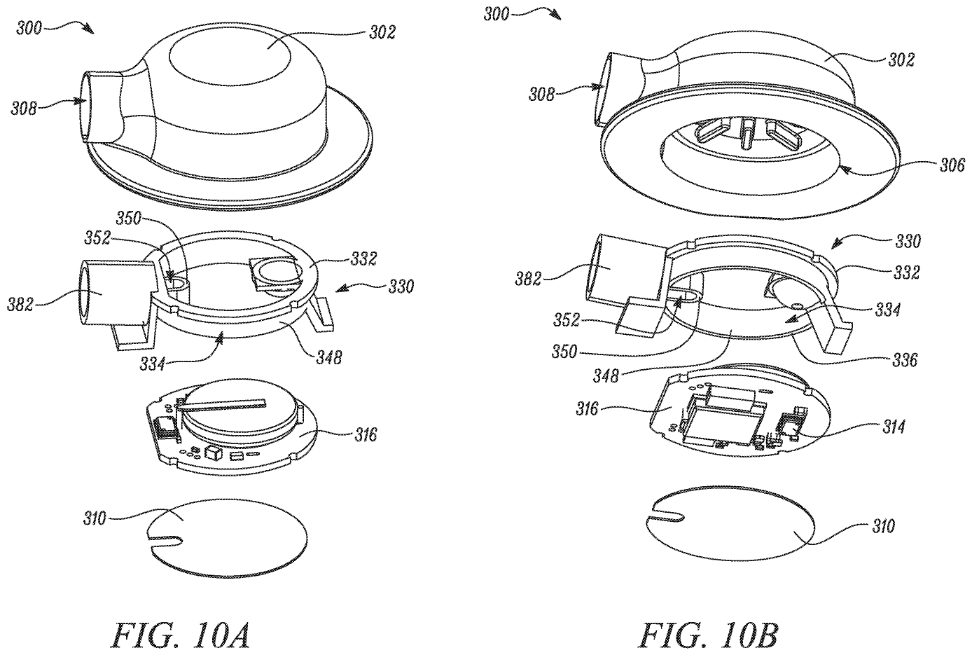

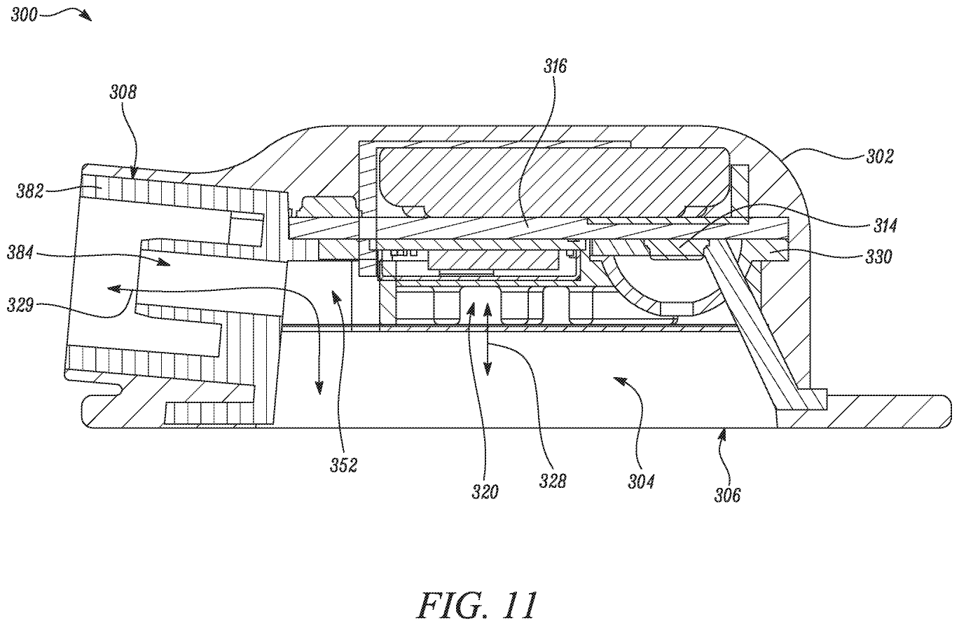

FIGS. 10A and 10B illustrate schematic top and bottom exploded perspective views, respectively, of the device 300. FIG. 11 is a schematic sectional view of the device 300 taken along a section line D-D′ shown in FIG. 9A. Referring to FIGS. 10A-11, the device 300 further includes a chamber 320 (shown in FIG. 11) defined between the housing 302 and the filter 310 opposite to the opening 306 (shown in FIG. 10B). An air flow 328 (shown by a double-headed arrow in FIG. 11) between the chamber 320 and the opening 306 occurs through the filter 310. The device 300 further includes a sensor 314 received within the chamber 320 and configured to sense at least one parameter associated with a wound site (e.g., the wound site 102 shown in FIG. 1).

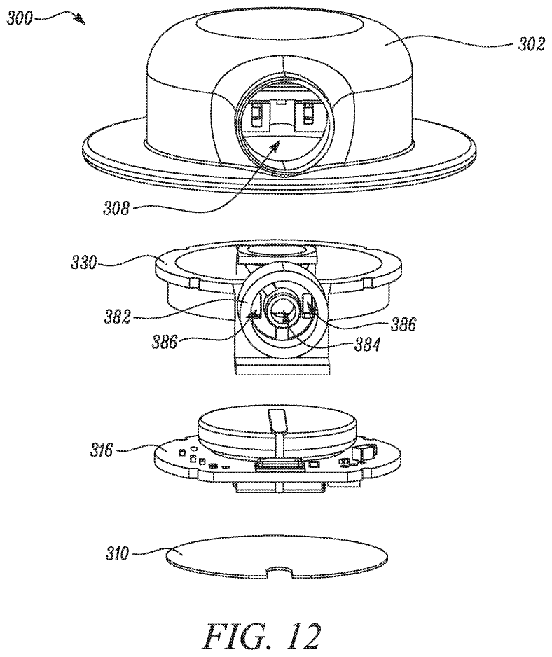

In some examples, the device 300 further includes a printed circuit board 316 received within the chamber 320 and coupled to the housing 302. In some examples, the printed circuit board 316 is disposed between the filter 310 and the housing 302 opposite to the opening 306. In some examples, the sensor 314 is coupled to and disposed on the printed circuit board 316, such that the sensor 314 faces the filter 310. In some examples, the device 300 further includes a carrier 330 received within the cavity 304 (shown in FIG. 12B) and removably coupled to the filter 310. In some examples, the carrier 330 at least partially engages with and is coupled to the housing 302. In some examples, the printed circuit board 316 is mounted on the carrier 330. In some examples, the carrier 330, the housing 302, and the filter 310 together define the chamber 320 therebetween.

In some examples, the carrier 330 includes a tubular portion 382 at least partially received within the housing port 308. In some examples, the tubular portion 382 is configured to be fluidly coupled to a conduit (e.g., the conduit 108 shown in FIG. 1) and receive a negative pressure from the negative pressure source.

In some examples, the carrier 330 further includes a base 332 engaging the printed circuit board 316 and a lateral wall 334 extending from the base 332 towards the opening 306. In some examples, the lateral wall 334 is continuous and surrounds the filter 310. In some examples, the lateral wall 334 sealingly engages the filter 310. Specifically, an end surface 336 (shown in FIG. 10B) of the lateral wall 334 sealingly engages the filter 310. In some examples, the lateral wall 334 further includes a circular portion 348 and a notch portion 350 connected to the circular portion 348 and curving radially inwards relative to the circular portion 348. In some examples, the notch portion 350 defines a recess 352 facing the tubular portion 382.

In some examples, the notch portion 350 and the recess 352 may allow the tubular portion 382 to fluidly communicate with the opening 306 while ensuring that the chamber 320 remains sealed at all times during application of the negative pressure to the wound site. In some examples, fluids may flow along a flow path 329 (shown by a curved double-headed arrow in FIG. 11) between the tubular portion 382 and the opening 306 away from the chamber 220.

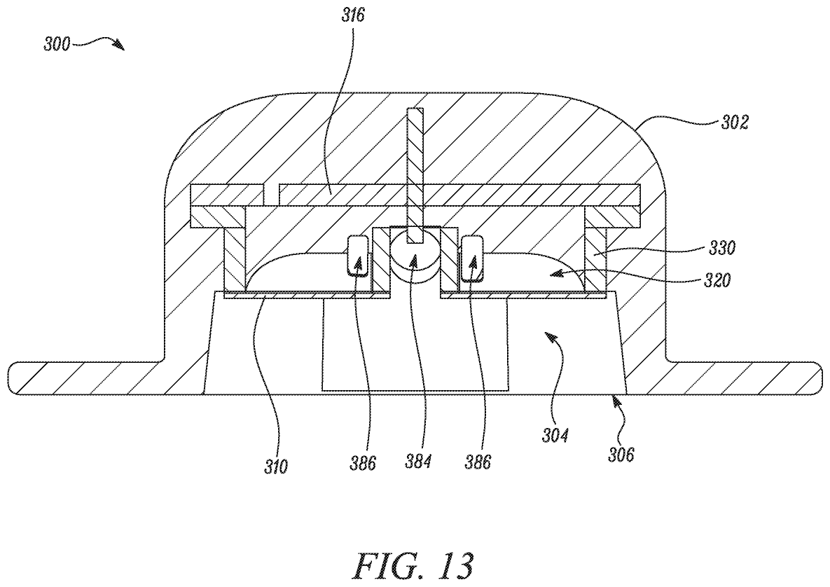

FIG. 12 illustrates a schematic front exploded perspective view of the device 300. FIG. 13 illustrates a schematic sectional view of the device 300 taken along a section line E-E′ shown in FIG. 9A. Referring to FIGS. 12 and 13, in some examples, the tubular portion 382 includes a negative pressure port 384 (also shown in FIG. 11) configured to fluidly communicate the negative pressure source with the cavity 304.

In some examples, the tubular portion 382 further includes one or more purge ports 386 configured to selectively fluidly communicate an ambient with the chamber 320. In some examples, the conduit (e.g., the conduit 108 shown in FIG. 1) may include a plurality of lumens. For example, the conduit may include a negative pressure lumen and one or more purge lumens. The negative pressure lumen may be fluidly couple the negative pressure port 384 to the negative pressure source. In some examples, the one or more purge lumens may fluidly couple a corresponding negative pressure port 384 to the ambient, e.g., via a purge valve within the negative pressure source.

In some examples, the one or more purge ports 386 are fluidly coupled to the chamber 320 only. Thus, the one or more purge ports 386 may selectively fluidly communicate the ambient with the chamber 320. In some examples, the one or more purge ports 386 may fluidly communicate the chamber 320 with the ambient when the filter 310 may be blocked, thereby allowing an air flow to be received within the chamber 320 and removing the blockages in the filter 310. In some examples, the purge valve may be controlled by a controller (e.g., the controller 112 shown in FIG. 1) to selectively fluidly communicate the ambient with the chamber 320.

In some examples, different sections of the filter 310 may be periodically cleaned via the one or more purge ports 386. For example, a quadrant of the filter 310 may be pneumatically exposed to one of the purge ports 386. The device 300 may clean one quadrant of the filter 310 at a time. Subsequently, another quadrant of the filter 310 may be cleaned. The one or more purge ports 386 may be controlled via a corresponding purge valve accordingly.

In some examples, the one or more purge ports 386 may allow a preventive maintenance for the filter 310 to be performed. Flow through the one or more purge ports 386 may be maximized when blockage is detected in the filter 310.

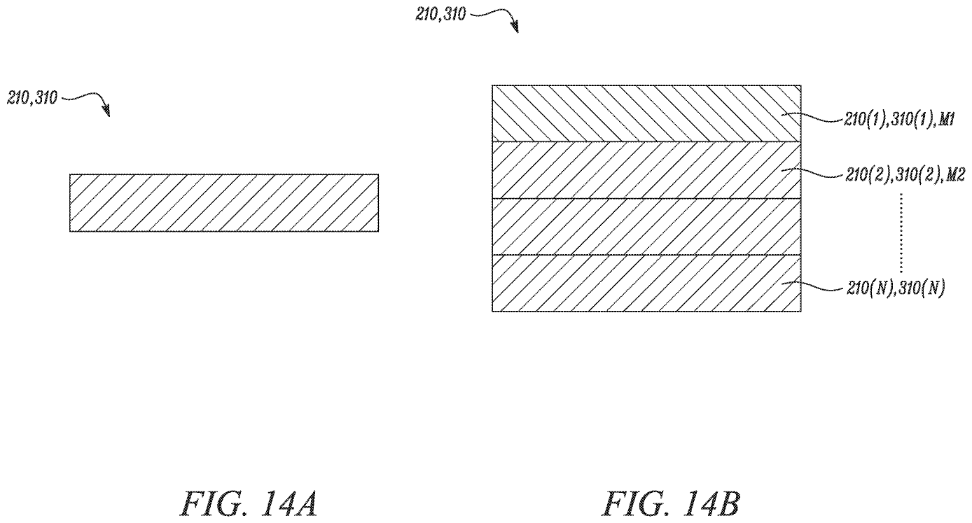

FIG. 14A is a schematic partial cross-sectional view of the filter 210, 310. In the illustrated embodiment of FIG. 14A, the filter 210, 310 is a single filter. In some examples, the filter 210, 310 may include any filtration medium. In some examples, the filter 210, 310 may be any means for separating or restricting substances. For example, the filter 210, 310 may separate gases or air from liquids or solids. In some examples, the filter 210, 310 may restrict flow of a liquid or a mixture of solids and liquids (e.g., wound exudates) therethrough while allowing flow of gases. In some examples, the filter 210, 310 may include poly-tetra-fluoro-ethylene (PTFE) woven fabric, such as, for example, a woven fiberglass membrane coated with PTFE.

FIG. 14B is a schematic partial cross-sectional view of the filter 210, 310, according to another embodiment of the present disclosure. In the illustrated embodiment of FIG. 14B, the filter 210, 310 includes a plurality of filters 210(1)-210(N), 310(1)-310(N) (collectively, filters 210, 310) stacked on each other, where N is a positive integer corresponding to a total number of the filters 210, 310 (e.g., N=3, 4, 5, etc.).

Referring to FIGS. 4, 11, and 14B, in some examples, the plurality of filters 210(1)-210(N), 310(1)-310(N) include a first filter 210(1), 310(1) facing the chamber 220, 320 and made of a first material M1. In some examples, the first material M1 includes a hydrophobic material or an oleophobic material. Thus, the first filter 210(1), 310(1) facing the chamber 220, 320 may prevent ingress of the wound exudates into the chamber 220, 320.

In some examples, the plurality of filters 210(1)-210(N), 310(1)-310(N) include a second filter 210(2), 310(2) disposed adjacent to the first filter 210(1), 310(1) away from the chamber 220, 320 and made of a second material M2 different from the first material M1. In some examples, the second material M2 includes a foam material (having various densities) or a polymeric material (e.g., a vacuum formed plastic film). Thus, second filter 210(2), 310(2) may reduce a volume of fluids contacting the first filter 210(1), 310(1). In some examples, the subsequent filters 210(3)-210(N), 310(3)-310(N) of the plurality of filters 210(1)-210(N), 310(1)-310(N) may be similar to the second filter 210(2), 310(2).

In some examples, the second filter 210(2), 310(2) is at least partially fenestrated. In other words, the second filter 210(2), 310(2) may have hole patterns, perforations, etc. which may offer benefits such as, but not limited to, increased air flow while reducing fluid flow, pressure manifolding, pressure offloading from sharp edges, etc.

In alternative embodiments, the plurality of filters 210(1)-210(N), 310(1)-310(N) are similar to each other and made of a same material. In such cases, the plurality of filters 210(1)-210(N), 310(1)-310(N) may offer redundancy if one or more filters 210, 310 from the plurality of filters 210(1)-210(N), 310(1)-310(N) are compromised.

Referring to FIGS. 1-13, the device 200, 300 of the present disclosure may not only protect the sensor 214, 314 as the sensor 214, 314 is received within the chamber 220, 320 defined between the housing 202, 302 and the filter 210, 310 opposite to the opening 206, 306, but also allow removal of any blockages from the filter 210, 310. In some embodiments, the filter 210, 310 may be periodically cleaned from any contaminants, thereby improving a performance of the sensor 214, 314 while also enhancing the service life of the sensor 214, 314. Further, this may allow continued sensing of the at least one parameter by the sensor 214, 314 over prolonged usage with the wound fluids. The sensor 214, 314 may continue to transmit accurate data associated with the wound site 102.

The filter 210, 310 may prevent the wound fluids from entering the chamber 220, 320, thereby protecting the sensor 214, 314. Further, the performance of the sensor 214, 314 may be prolonged by eliminating the blockages from the filter 210, 310. Thus, the device 200, 300 of the present disclosure may allow the sensor 214, 314 to perform intended functions accurately throughout the application of the negative pressure therapy to the wound site 102. Additionally, the filter 210, 310 being air permeable may allow the sensor 214, 314 to perform the intended functions while being enclosed within the chamber 220, 320.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A device for interfacing a negative pressure source with a dressing disposed on a wound site, the device comprising:
a housing comprising:
a cavity comprising an opening configured to fluidly communicate with the dressing; and
a housing port extending through the housing and fluidly communicating with the cavity, wherein the housing port is configured to fluidly couple the cavity with the negative pressure source;
a filter at least partially covering the cavity of the housing and facing the opening of the housing, wherein the filter is air permeable;
a chamber defined between the housing and the filter opposite to the opening, wherein an air flow between the chamber and the opening occurs through the filter; and
a sensor received within the chamber and configured to sense at least one parameter associated with the wound site.

2. The device of claim 1, further comprising a printed circuit board received within the chamber and coupled to the housing, wherein the printed circuit board is disposed between the filter and the housing opposite to the opening, and wherein the sensor is coupled to and disposed on the printed circuit board, such that the sensor faces the filter.

3. The device of claim 2, further comprising a carrier received within the cavity and removably coupled to the filter, wherein the carrier at least partially engages with and is coupled to the housing, wherein the printed circuit board is mounted on the carrier, and wherein the carrier, the housing, and the filter together define the chamber therebetween.

4. The device of claim 3, wherein the carrier comprises a base engaging the printed circuit board and a lateral wall extending from the base towards the opening, wherein the lateral wall is continuous and surrounds the filter, and wherein the lateral wall sealingly engages the filter.

5. The device of claim 3, wherein the carrier comprises a tubular portion at least partially received within the housing port, and wherein the tubular portion comprises:
a negative pressure port configured to fluidly communicate the negative pressure source with the cavity; and
one or more purge ports configured to selectively fluidly communicate an ambient with the chamber.

6. The device of claim 1, wherein the housing further comprises at least one leak path extending therethrough and fluidly communicating with the chamber.

7. The device of claim 6, further comprising an ambient carrier coupled to the housing and disposed opposite to the filter, such that the chamber is disposed between the ambient carrier and the filter, wherein the ambient carrier comprises at least one ambient opening extending therethrough, and wherein each of the at least one ambient opening is disposed in fluid communication with the corresponding at least one leak path.

8. The device of claim 7, further comprising an ambient filter disposed on the ambient carrier opposite to the at least one leak path and removably coupled to the housing, wherein the ambient filter covers the at least one ambient opening of the ambient carrier and is exposed to an ambient, and wherein the ambient filter is air permeable.

9. The device of claim 7, wherein the ambient carrier further comprises:
a main body, wherein the at least one ambient opening extends through the main body; and
at least one hollow tube extending from the main body, wherein each of the at least one hollow tube is at least partially received within and disposed in fluid communication with the corresponding at least one leak path, and wherein each of the at least one hollow tube is disposed in fluid communication with the corresponding at least one ambient opening.

10. The device of claim 9, wherein the ambient carrier further comprises a tube wall disposed within each of the at least one hollow tube and comprising an orifice extending therethrough, and wherein each of the at least one hollow tube fluidly communicates the corresponding at least one leak path with the corresponding at least one ambient opening via the corresponding orifice.

11. The device of claim 9, further comprising a check valve disposed in each of the at least one hollow tube, wherein the check valve opens and allows a unidirectional flow from the corresponding at least one ambient opening to the chamber when a pressure difference across the check valve is below a threshold pressure difference, and wherein the check valve substantially closes and allows a leakage flow therethrough when the pressure difference across the check valve is greater than or equal to the threshold pressure difference.

12. The device of claim 9, further comprising a check valve disposed in each of the at least one hollow tube, wherein the check valve opens and allows a unidirectional flow from the corresponding at least one ambient opening to the chamber when a pressure difference across the check valve is above a threshold pressure difference, and wherein the check valve substantially closes and allows a leakage flow therethrough when the pressure difference across the check valve is less than or equal to the threshold pressure difference.

13. The device of claim 6, wherein the at least one leak path comprises a plurality of leak paths spaced apart from each other.

14. The device of claim 1, wherein the sensor is a pressure sensor, and wherein the at least one parameter is a negative pressure within the chamber.

15. A system for negative pressure therapy of a wound site, the system comprising:
a dressing disposed on the wound site;
a negative pressure source; and
a device for interfacing the negative pressure source with the dressing, the device comprising:
a housing disposed on the dressing, the housing comprising:
a cavity comprising an opening fluidly communicating with the dressing; and
a housing port extending through the housing and fluidly communicating with the cavity, wherein the housing port fluidly couples the cavity with the negative pressure source;
a filter at least partially covering the cavity of the housing and facing the opening of the housing, wherein the filter is air permeable;
a chamber defined between the housing and the filter opposite to the opening, wherein an air flow between the chamber and the opening occurs through the filter; and
a sensor received within the chamber and configured to sense at least one parameter associated with the wound site.

16.-19. (canceled)

20. The system of claim 15, wherein the housing further comprises at least one leak path extending therethrough and fluidly communicating with the chamber.

21. The system of claim 20, further comprising an ambient carrier coupled to the housing and disposed opposite to the filter, such that the chamber is disposed between the ambient carrier and the filter, wherein the ambient carrier comprises at least one ambient opening extending therethrough, and wherein each of the at least one ambient opening is disposed in fluid communication with the corresponding at least one leak path.

22. (canceled)

23. The system of claim 21, wherein the ambient carrier further comprises:
a main body, wherein the at least one ambient opening extends through the main body; and
at least one hollow tube extending from the main body, wherein each of the at least one hollow tube is at least partially received within and disposed in fluid communication with the corresponding at least one leak path, and wherein each of the at least one hollow tube is disposed in fluid communication with the corresponding at least one ambient opening.

24. (canceled)

25. (canceled)

26. The system of claim 23, further comprising a check valve disposed in each of the at least one hollow tube, wherein the check valve opens and allows a unidirectional flow from the corresponding at least one ambient opening to the chamber when a pressure difference across the check valve is above a threshold pressure difference, and wherein the check valve substantially closes and allow a leakage flow therethrough when the pressure difference across the check valve is less than or equal to the threshold pressure difference.

27. The system of claim 26, further comprising a controller communicably coupled to the negative pressure source and the sensor, wherein the controller is configured to:
control the negative pressure source to maintain a negative pressure within the dressing at a predetermined negative pressure, wherein the pressure difference across the check valve is less than the threshold pressure difference when the negative pressure within the dressing is equal to the predetermined negative pressure;
determine the negative pressure within the chamber based on a signal received from the sensor;
determine that the filter is blocked when the negative pressure within the chamber falls below the predetermined negative pressure; and
control the negative pressure source to increase the negative pressure within the dressing above the predetermined negative pressure, such that the pressure difference across the check valve is greater than the threshold pressure difference.

28. (canceled)

29. (canceled)
